(19) United States
(12) Patent Application Publication
(10) Pub. No.: US 2017/0182128 A1
(43) Pub. Date: Jun. 29, 2017

(54) METHOD OF TREATING HEART FAILURE

(71) Applicant: University of Pittsburgh - Of the Commonwealth System of Higher Education, Pittsburgh, PA (US)

(72) Inventors: David Schwartzman, Pittsburgh, PA (US); Sanjeev G. Shroff, Pittsburgh, PA (US); Guy Salama, Pittsburgh, PA (US); Charles McTiernan, Pittsburgh, PA (US)

(73) Assignee: University of Pittsburgh - Of the Commonwealth System of Higher Education, Pittsburgh, PA (US)

(21) Appl. No.: 15/456,184

(22) Filed: Mar. 10, 2017

Related U.S. Application Data
(63) Continuation of application No. 12/722,386, filed on Mar. 11, 2010, now abandoned.
(60) Provisional application No. 61/160,047, filed on Mar. 13, 2009.

(51) Int. Cl. A61K 38/22

(52) U.S. Cl. CPC A61K 38/2221

(57) ABSTRACT

The present invention includes methods, systems, uses, and means for the delivery of one or more anti-fibrotic agents into the pericardial space for the treatment and prevention of heart failure (HF).

### CROSS REFERENCE TO RELATED APPLICATIONS

This application is a continuation of U.S. application Ser. No. 12/722,386, filed Mar. 11, 2010, which in turn claims the benefit of U.S. Provisional Application No. 61/160,047, filed Mar. 13, 2009; each of these applications is specifically incorporated by reference herein in its entirety.

### BACKGROUND

Heart failure (HF) is a common cardiovascular condition where the heart cannot circulate enough blood and oxygen to meet the needs of other body organs. Heart failure is a serious condition and there is currently no cure. Once diagnosed, medicines are needed for the rest of the person's life. Around 5 million people in the United States have heart failure, with about 550,000 new cases diagnosed each year. More than 287,000 people in the United States die each year with heart failure.

Hospitalizations for heart failure have increased substantially, rising from 402,000 in 1979 to 1,101,000 in 2004, and heart failure is the most common reason for hospitalization among people on Medicare (Centers for Disease Control and Prevention (CDC) “Heart Failure Fact Sheet,” September 2006; available on the worldwide web at cdc.gov/dhdsp/library/fs_heart_failure.htm). Since heart failure is more common in the elderly, its prevalence is likely to continue to increase as the population ages, and is expected to double within the next thirty years. Heart failure is increasing in prevalence and incidence and represents a major public health problem in industrialized nations.

Ejection fraction (EF) is the percentage of blood pumped out of the left ventricle with each heartbeat. Ejection fraction may be measured, for example, during an echocardiogram. Ejection fraction is an important measurement of how well a heart is pumping and can be used to classify heart failure and to guide treatment. Clinicians and researchers are focusing increasingly on the distinction between reduced ejection fraction and preserved ejection fraction in patients with heart failure. Heart failure can be classified or as heart failure with reduced ejection fraction (also referred to as systolic heart failure).

A recent study demonstrated that the prevalence of heart failure with preserved ejection fraction increased over a 15-year period, with no marked improvement in the mortality rates. If these trends continue, heart failure with preserved ejection fraction may become the most common form of heart failure, demonstrating a growing public health problem. See “Trends in prevalence and outcome of heart failure with preserved ejection fraction,” Owan et al., 2006, N Engl J Med; 355 (3):251-9. There are currently no proven therapies for the treatment of heart failure with preserved ejection fraction, and the treatment of heart failure with preserved ejection fraction represents a significant unmet medical need.

### SUMMARY OF THE INVENTION

The present invention includes methods of treating heart disease with the pericardial administration of an anti-fibrotic agent. In some aspects of the methods of the present invention, heart disease includes heart failure or atrial fibrillation, ventricular fibrillation and other arrhythmias. In some aspects, pericardial administration of an anti-fibrotic agent includes advancing an infusion catheter to dispose a distal catheter segment having a distal infusion catheter lumen end opening in the pericardial space; attaching a proximal connector of the infusion catheter to an infusion pump having a reservoir including the anti-fibrotic agent; and delivering the anti-fibrotic agent from the reservoir into the pericardial space. In some aspects of the methods of the present invention, the method further includes detecting a condition of the heart and delivering an effective amount of the anti-fibrotic agent. In some aspects, the methods of the present invention further include detecting a remotely transmitted therapy delivery command, detecting a measurement of the heart failure state of the heart, and/or delivering an effective amount of the anti-fibrotic agent.

The present invention includes methods of transvenously accessing the pericardial space between a heart and its pericardium to deliver a pharmacologic agent to the heart to treat heart disease, the method including passing a fixation catheter having a fixation catheter lumen extending between proximal and distal fixation catheter lumen openings and a distal tissue fixation mechanism through a selected peripheral vein and one of the inferior vena cava and the superior vena cava to establish a transvenous route into the right atrium of the heart; disposing the distal fixation mechanism and distal fixation catheter lumen opening proximate the right atrial wall; affixing the distal fixation mechanism to the right atrial wall; passing an infusion catheter through the fixation catheter lumen out of the distal fixation catheter lumen opening and through the stabilized atrial wall to dispose a distal catheter segment having a distal infusion catheter lumen end opening in the pericardial space; and delivering a pharmacologic agent through the infusion catheter to treat heart failure. In some aspects, the delivering step includes attaching a proximal connector of the infusion catheter to an infusion pump; subcutaneously implanting the infusion pump in the thoracic region; and operating the infusion pump to deliver the pharmacologic agent into the pericardial space. In some aspects, the operating step includes detecting a remotely transmitted therapy delivery command; and delivering a bolus including the pharmacologic agent. In some aspects, heart disease includes heart failure or atrial fibrillation, ventricular fibrillation and other arrhythmias.

The present invention includes methods of accessing the pericardial space between a heart and its pericardium to deliver an anti-fibrotic agent to the heart, the method including advancing an infusion catheter to dispose a distal catheter segment having a distal infusion catheter lumen end opening in the pericardial space; attaching a proximal connector of the infusion catheter to an infusion pump; detecting a condition of the heart; and delivering an effective amount of the anti-fibrotic agent. In some aspect, the heart condition includes heart failure or atrial fibrillation, ventricular fibrillation and other arrhythmias.

The present invention includes a system for delivering an anti-fibrotic agent into the pericardial space surrounding the heart to treat heart disease. In some aspects, heart disease includes heart failure or atrial fibrillation, ventricular fibrillation and other arrhythmias. In some aspects, the system includes an infusion pump having a reservoir having an anti-fibrotic agent to be delivered into the pericardial space; and an infusion catheter coupled a proximal catheter end to the infusion pump and adapted to be routed to dispose a distal catheter segment having a distal infusion catheter lumen end opening in the pericardial space. In some aspects of the system, the infusion pump further includes a means for detecting a condition of the heart and a means for delivering an amount of the anti-fibrotic agent into the pericardial space effective to counter the detected heart condition. In some aspects of the system, the infusion pump further includes a means for regulating the delivery of the anti-fibrotic agent into the pericardial space to counter the detected condition. In some aspects of the system, the infusion pump further includes a means responsive to a remotely transmitted therapy delivery command for delivering the anti-fibrotic agent.

The present invention includes the use of an anti-fibrotic agent in the manufacture of a medicament for pericardial administration for the treatment of a heart disease. In some aspects, heart disease includes heart failure or atrial fibrillation, ventricular fibrillation and other arrhythmias.

The present invention includes means for the pericardial delivery of an anti-fibrotic agent for the treatment of heart disease. In some aspects, heart disease includes heart failure or atrial fibrillation, ventricular fibrillation and other arrhythmias.

In some aspects of the methods, systems, uses, or means of the present invention, pericardial administration of an anti-fibrotic agent includes subxyphoid delivery, an epicardial catheter, a transarterial catheter, an external drug delivery system, and/or an implantable drug delivery system. In some aspects of the present invention, pericardial administration of an anti-fibrotic agent includes an epicardial catheter and includes an external drug delivery system and/or an implantable drug delivery system. In some aspects of the present invention, pericardial administration of an anti-fibrotic agent includes a transarterial catheter and includes an external drug delivery system and/or an implantable drug delivery system. In some aspects of the present invention, pericardial administration of an anti-fibrotic agent includes subxyphoid delivery and includes an external drug delivery system and/or an implantable drug delivery system.

In some aspects of the methods, systems, uses, or means of the present invention, heart disease includes heart failure. In some aspects of the methods, systems, uses, or means of the present invention, heart failure includes heart failure with preserved ejection fraction.

In some aspects of the methods, systems, uses, or means of the present invention, the anti-fibrotic agent includes an ACE inhibitor, a lectin, sirolimus, tacrolimus, pirfenidone, a small molecule relaxin agonist, relaxin, or combinations thereof. In some aspects of the methods, systems, uses, or means of the present invention, the anti-fibrotic agent includes relaxin.

In some aspects of the methods, systems, uses, or means of the present invention, the method, system, use, or means further includes detecting one or more measurements of the state of the heart. In some aspects, one or more measurements are detected remotely. In some aspects, the method, system, or use further includes delivering an effective amount of the anti-fibrotic agent. In some aspects, the method, system, use, or means further includes detecting a remotely transmitted therapy delivery command; and delivering a bolus including the pharmacologic agent.

The terms “comprises” and variations thereof do not have a limiting meaning where these terms appear in the description and claims.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

Unless otherwise specified, “a,” “an,” “the,” “at least one,” and “one or more” are used interchangeably. The term “and/or” means one or all of the listed elements/characteristics or a combination of any two or more of the listed elements/characteristics. As used herein, the term “or” is generally employed in its usual sense including “and/or” unless the content clearly dictates otherwise.

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

For any method disclosed herein that includes discrete steps, the steps may be conducted in any feasible order. And, as appropriate, any combination of two or more steps may be conducted simultaneously.

### DETAILED DESCRIPTION OF ILLUSTRATIVE EMBODIMENTS OF THE INVENTION

The present invention includes methods, systems, uses and means for the delivery of one or more therapeutic agents into the pericardial space for the treatment and prevention of heart diseases, including, but not limited to, heart failure and ventricular fibrillation, atrial fibrillation, and other arrhythmias. Heart failure (HF) includes, but is not limited to, heart failure with preserved ejection fraction and heart failure with reduced ejection fraction. The targeted pericardial delivery of agent of the present invention represents a major advance in the treatment of heart disease, allowing for the delivery of therapeutic concentrations of therapeutic agents directly to the heart and avoiding the sometimes serious side effects observed with the systemic delivery of such agents.

In some embodiments, the methods of the present invention are used for the treatment of heart failure. Heart failure is the clinical syndrome that can result from any structural or functional cardiac disorder that impairs the ability of the ventricle to fill with or eject blood. In a healthy heart, the ejection fraction (EF) is about 60 percent, meaning that about 60 percent of the blood that fills the left ventricle is pumped out with each beat. Heart failure may be associated with reduced pumping function as measured by the ejection fraction (referred to herein as heart failure with reduced ejection fraction or systolic heart failure) or with reduced relaxing function with preserved ejection fraction (referred to herein as heart failure with preserved ejection:fraction or diastolic heart failure). These two types of heart dysfunction exhibit similar symptoms. Measurement of heart function, for example, with an echocardiogram, may be used to distinguish between the two forms of heart failure.

The methods of the present invention may be used for the treatment of heart failure with reduced ejection fraction and/or for the treatment of heart failure with preserved ejection fraction.

In a preferred embodiment, the methods of the present invention may be used for the treatment of heart failure with preserved ejection fraction. Heart failure with preserved ejection fraction may be caused, for example, by increased resistance to ventricular inflow and reduced ventricular diastolic capacity due to reduced compliance or stiffness (constrictive pericarditis and restrictive, hypertensive, and hypertrophic cardiomyopathy), impaired ventricular relaxation (acute myocardial ischemia), and myocardial fibrosis and infiltration (restrictive cardiomyopathy). Heart failure with preserved ejection fraction may be represented by an ejection fraction of about 50 percent or greater. In some embodiments, heart failure with reduced ejection fraction may be represented by an ejection fraction of less than about 50 percent.

With the methods and systems of the present invention, one or more anti-fibrotic agents may be delivered to the pericardial space. Anti-fibrotic agents may include, but are not limited to, angiotensin converting enzyme (ACE) inhibitors, lectins, sirolimus, tacrolimus, pirfenidone, small molecule relaxin agonists, such as, for example, H2 relaxin receptor agonists, and relaxin. ACE inhibitors, include, but are not limited to, benazepril, captopril, enalapril, fosinopril, imidapril, lisinopril, moexipril, quinapril, perindopril, erbumine, ramipril, and trandolapril. Lectins bind to carbohydrate moieties and include, but are not limited to, lectins which bind to N-acetylglucosamine, such as chitin-binding lectins and mannose-binding lectins and any of those described in U.S. Pat. No. 7,026,287. A lectin may be derived from plant or animal species, or synthesized by a bioprocess, such as fermentation. Pirfenidone [5-methyl-1-phenyl-2-(1H)-pyridone] is a small molecule drug that inhibits collagen synthesis, down regulates profibrotic cytokines, and decreases fibroblast proliferation. Pirfenidone has recently been approved for use in idiopathic pulmonary fibrosis (IPF) patients in Japan and is marketed as Pirespa® by Shionogi in that country. Sirolimus, a macrocyclic lactone produced by Streptomyces hygroscopicus, is also known as rapamycin and is marketed as Rapamune® by Wyeth. Tacrolimus, a 23-membered macrolide lactone produced by Streptomyces tsukubaensis, is also known as FK-506 or Fujimycin and is marketed under the tradenames Prograf, Advagraf, and Protopic by Astellas Pharma Inc.

In preferred embodiments, the anti-fibrotic agent is relaxin, an analog thereof, or a variant thereof. Relaxin (“RLX”) is a naturally occurring peptide hormone that plays an important physiological role within the body to orchestrate many of the maternal physiological responses to pregnancy. It is well established that relaxin's ability to regulate collagen turnover is essential for softening the pelvic ligaments and female reproductive organs in preparation for child birth (Hisaw, 1926, Pro Soc Exp Bio. Med; 23:661-663; Schwabe et al., 1977, Biochem Biophys Res Comm; 75:503-510; James et al., 1977, Nature; 267:544-546). In addition to acting on the female reproductive system, relaxin also affects non-reproductive targets, including the cardiovascular system and the connective tissue.

Relaxin is a member of a peptide hormone family that diverged from insulin early in vertebrate evolution and has been assigned to a specific hormone family, termed the relaxin peptide family. The relaxin peptide family includes three different relaxins, relaxin-1, relaxin-2 and relaxin-3, as well as insulin-like peptide (INSL)3, INSL4, INSL5 and INSL6. All share high structural similarity with insulin due to the presence of six cysteine residues, which confer two inter-chain and one intra-chain disulfide bonds. Three relaxin genes are present in humans. Relaxin-1 is found only in humans and the great apes and its expression is limited to the decidua, placenta and prostate. Relaxin-2 is the major circulating form of relaxin in the human and the functional equivalent to the relaxin-1 in all non-primates. Relaxin-3 has only recently been discovered and shows brain specific expression. Circulating relaxin accounts for most of the known biological effects of the hormone in humans and experimental animals. See, for example, Bani, 2008, Vasc Health Risk Manag; 4 (3):515-524. As used herein, relaxin includes relaxin-2 found in humans and great apes. In some embodiments of the present invention, relaxin includes relaxion-1, relaxin-2 and/or relaxin 3.

Like insulin, the structure of relaxin is formed by the cleavage of a pro-hormone peptide into three chains (A, B and C), the removal of the C chain and the formation of three disulfide bridges between six invariant cysteine residues found on the A and B chains, to produce an active protein. Structurally, relaxin is composed of A and B chains stabilized by inter- and intra-domain disulfide bonds with a molecular weight of approximately 6000 daltons.

Relaxin for use in the methods and systems of the present invention includes, but is not limited to, relaxin of a variety of species, including, but not limited to, porcine, murine, equine, shark, tiger, rat, dogfish, and human relaxin. The complete amino acid sequences and DNA sequences encoding the relaxin polypeptide are known for a variety of species, including human relaxin (see, for example, Hudson et al., 1983, Nature; 301, 628-631; Hayes, 2004, Reprod Biol Endocrinol; 2:36; Sherwood, 2004, Endocr Rev; 25 (2):205-34; and Wilkinson et al., 2005, BMC Evolutionary Biology; 5:14).

Relaxin includes relaxin isolated from native sources and relaxin produced using recombinant techniques, or chemically or enzymatically synthesized. In a preferred embodiment, relaxin is human relaxin, including, but not limited to, recombinant human relaxin (“rhRLX”) (R&D Systems®, Minneapolis, Minn. and Corthera Inc., San Mateo, Calif.).

Relaxin analogs may be used in the methods and systems of the present invention. Such analogs may include, for example, the relaxin analog B-R13/17K H2 (Hossain et al. “The chemically synthesized human relaxin-2 analog, B-R13/1 7K H2, is an RXFP1 antagonist,” Amino Acids, 2009 Dec 31 Epub ahead of print) and cyclic and linear relaxin peptide mimetics (Hossain et al., 2009 NY Acad Sci; 1160:16-19). Relaxin variants may include, for example, relaxin chimeras (Haugaard-Jons son et al., 2009, NY Acad Sci; 1160:27-30).

Through the use of recombinant DNA technology, relaxin variants may be prepared by altering the underlying DNA. All such variations or alterations in the structure of the relaxin molecule resulting in variants are included within the scope of this invention. Such variants include insertions, substitutions, or deletions of one or more amino acid residues, glycosylation variants, unglycosylated relaxin, organic and inorganic salts, covalently modified derivatives of relaxin, preprorelaxin, and prorelaxin. Such variant may maintain one or more of the functional, biological activities of the relaxin polypeptide. Variants of relaxin having such functional, biological activities can be readily identified using known in vitro or in vivo assays, such as any of those described in U.S. Pat. No. 5,945,402 and Lekgabe et al., 2005, Hypertension; 46:412-418. An anti-fibrotic agent of the present invention may be modified, for example, by PEGylation, to increase the half life of the anti-fibrotic agent in the recipient, to retard clearance from the pericardial space, and/or to make the anti-fibrotic agent more stable for delivery by a pump.

Agents to be administered in the methods of the present invention include, but are not limited to, anti-fi.brotic agents. In some aspects of the present invention, the agent is not an anti-fi.brotic agent. Such agents include, but are not limited to, genetic agents, biologic agents, and pharmacologic agents. Suitable pharmacologic agents include, for example, agents that are angiotensin receptor blockers (ARB), including, but not limited to, candesartan (Atacand), eprosartan (Teveten), irbesartan (Avapro), telmisartan (Micardis), valsartan (Diovan), losartan (Cozaar), and olmesartan (Benicar), agents that angiotensin converting enzyme (ACE) inhibitors, including, but not limited to, benazepril (Lotensin), captopril (Capoten), enalapril (Vasotec), fosinopril (Monopril), lisinopril (Prinivil, Zestril), moexipril (Univasc), perindopril (Aceon), quinapril (Accupril), ramipril (Altace) and trandolapril (Mavik), agents that improve cardiac contractility (including, but not limited to, digitalis drugs and adrenergic agonists), agents that suppress arrhythmias (including, but not limited to, class I, II, III, and IV agents and specialized drugs such as amiodarone), agents that dilate coronary arteries (including, but not limited to, nitroglycerin and calcium channel blockers), agents that lyse clots in the coronary circulation (including, but not limited to, thrombolytic agents such as streptokinase or tissue-type plasminogen activator (TPA)), agents that reverse symptoms of heart failure (including, but not limited to, beta-adrenergic blockers) and vasodilator, antiplatelet, anticoagulant, thrombolytic, anti-inflammatory, antiarrhythmic, initropic, antimitotic, angiogenic, and antiatherogenic agents.

Agents may be formulated for delivery to the pericardial space according to any of a variety of methods known and used in the art, based on several variables, including, for example, the specific condition being treated, the recipient's medical history, and the therapeutic route and schedule being utilized. Agents may be formulated for administration to the pericardial space as a composition. A composition may also include, for example, one or more accessory ingredients including, but not limited to, diluents, buffers, binders, disintegrants, surface active agents, thickeners, lubricants, gels, colloids, emulsifiers, preservatives, including, for example, antioxidants, and the like. A composition may include a pharmaceutically acceptable carrier. The term “pharmaceutically acceptable,” as used herein, means that the compositions or components thereof so described are suitable for use in contact with the tissues of humans and animals without undue toxicity, irritation, allergic response, and the like. The preparation of such compositions is well understood in the art. Compositions for pericardial administration may be in an aqueous solution. A composition may be sterile and may be suitably buffered if necessary. A composition may be pyrogen free, assayed, for example, in a Limulus amoebocyte lysate (LAL) assay. A composition may be rendered isotonic with the recipient's blood or plasma with sufficient saline or glucose. In some embodiments, a composition may be in a formulation for controlled, sustained, or extended release. Many such suitable formulations are known, including, for example, polymeric or protein microparticle formulations.

Anti-fibrotic agents may be administered as compositions including one or more isolated anti-fibrotic agents. As used herein, the term isolated means a preparation that is either removed from its natural environment or synthetically derived, for instance by recombinant techniques, or chemically or enzymatically synthesized. In a preferred form, an isolated anti-fibrotic agent is purified and substantially free of other agents. The present invention also includes compositions including two or more anti-fibrotic agents.

Suitable dosing regimens may be determined by taking into account factors well known in the art including, for example, the age, weight, sex, and medical condition of the subject, the route of administration, the desired effect, and the particular conjugate and formulation employed. Therapeutically effective concentrations and amounts may be determined for each application herein empirically by testing the compounds in known in vitro and in vivo systems, including, but not limited to, any of those described herein; dosages for humans or other animals may then be extrapolated therefrom. The efficacy of treatment may be assessed by any of various parameters well known in the art. Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by the FDA and other regulatory agencies.

It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions and methods.

By a “therapeutically effective amount” of an anti-fibrotic agent is meant a sufficient amount of the compound to treat the subject at a reasonable benefit/risk ratio applicable to obtain a desired therapeutic response. It will be understood, however, that the total daily usage of the compounds and compositions of the present invention will be decided by the attending physician within the scope of sound medical judgment. The specific therapeutically effective dose level for any particular patient will depend upon a variety of factors including, for example, the disorder being treated and the severity of the disorder, activity of the specific compound employed, the specific composition employed, the age, body weight, general health, sex and diet of the patient, the time of administration, route of administration, and rate of excretion of the specific compound employed, the duration of the treatment, drugs used in combination or coincidentally with the specific compound employed, and like factors well known in the medical arts. Total daily dose of the compounds of this invention administered to a host in single or divided doses may be in amounts as determined by the attending physician.

The targeted pericardial delivery of agent of the present invention represents a major advance in the treatment of heart disease, allowing for the delivery of therapeutic concentrations of therapeutic agents directly to the heart and avoiding the sometimes serious side effects observed with the systemic delivery of such agents. While multiple studies have demonstrated that relaxin is safe for systemic administration (see, for example, Chen et al., 1993, Pharm Res; 10 (6):834-8; Seibold et al., 1998, J Rheumatol; 25 (2):302-7; Seibold et al., 2000, Ann Intern Med; 6:132 (11):871-9), a recent study demonstrates the potential for sometimes serious side effects with the systemic delivery of relaxin. In this large, randomized, double-blind, placebo-controlled phase III study, recombinant human relaxin (10 μg/kg/day or 25 μg/kg/day), or a placebo were administered by continuous subcutaneous infusion over 24 weeks to patients with systemic sclerosis (scleroderma), a disease characterized by fibrosis of the skin, vasculature, and internal organs. With this study, the systemic administration of relaxin was associated with serious renal adverse events, including doubling of serum creatinine, renal crisis, and grade 3-4 hypertension, especially after cessation of the infusion. The authors advise that “if relaxin is used therapeutically for any conditions other than scleroderma, close monitoring of blood pressure and renal function must be performed. See Khanna et al., 2009, Arthritis Rheum; 60 (4):1102-11.

In some embodiments, an anti-fibrotic agent may be administered to the pericardial space at a dosage that is equivalent to a dose of at least about 0.001 μg/kg/day, at least about 0.0025 μg/kg/day, at least about 0.005 μg/kg/day, of at least about 0.01 μg/kg/day, at least about 0.025 μg/kg/day, at least about 0.05 μg/kg/day, at least about 0.1 μg/kg/day, at least about 0.25 μg/kg/day, at least about 0.5 μg/kg/day, at least about 1 μg/kg/day, at least about 2.5 μg/kg/day, at least about 5 μg/kg/day, at least about 10 μg/kg/day, at least about 25 μg/kg/day, at least about 50 μg/kg/day, at least about 100 μg/kg/day, at least about 250 μg/kg/day, at least about 0.5 mg/kg/day, at least about 1 mg/kg/day, at least about 5 mg/kg/day, at least about 10 mg/kg/day, at least about 25 mg/kg/day, at least about 50 mg/kg/day, at least about 100 mg/kg/day, of at least about 250 mg/kg/day, or at least about 500 mg/kg/day. In some embodiments, an anti-fibrotic agent may be administered to the pericardial space at a dosage of at most about 0.001 μg/kg/day, of at most about 0.0025 μg/kg/day, of at most about 0.005 μg/kg/day, of at most about 0.01 μg/kg/day, of at most about 0.025 μg/kg/day, of at most about 0.05 μg/kg/day, of at most about 0.1 μg/kg/day, of at most about 0.25 μg/kg/day, of at most about 0.5 μg/kg/day, of at most about 1 μg/kg/day, of at most about 2.5 μg/kg/day, of at most about 5 μg/kg/day, of at most about 10 μg/kg/day, of at most about 25 μg/kg/day, of at most about 50 μg/kg/day, of at most about 100 μg/kg/day, of at most about 250 μg/kg/day, of at most about 500 μg/kg/day, at most about 1 mg/kg/day, at most about 2.5 mg/kg/day, at most about 5 mg/kg/day, at most about 10 mg/kg/day, at most about 25 mg/kg/day, at most about 50 mg/kg/day, at most about 100 mg/kg/day, at most about 250 mg/kg/day, or at most about 500 mg/kg/day. In some embodiments, an anti-fibrotic agent may be administered to the pericardial space in a range of any two of the above dosages.

Because of the focused mode of deliver provided by the present invention, in some embodiments an effective dosage delivered to the pericardial space may be less than the effective dosage utilized when the same agent is delivered systemically, such as for example, delivered intravenously or subcutaneously. In some embodiments, the effective dosage for pericardial delivery may be approximately ten-fold lower, approximately hundred-fold lower, approximately thousand-fold lower, or less, than the dosage for systemic delivery of the same anti-fibrotic agent. Delivery of an anti-fibrotic agent may be acute (as a single administration), subacute (delivered over days or weeks), or a long term, chronic delivery.

With the present invention, the pericardial delivery of an anti-fibrotic agent may be continuous or may be intermittent. Intermittent delivery may occur at predetermined timed intervals over the entire twenty-four hour day, for example, once a day, twice a day, three.times a day, four times a day, six times a day, eight times a day, twelve times a day, or twenty-four times a day. A baseline dosage, delivered continuously or intermittently at specified intervals, may be supplemented with a bolus dosage. Such a bolus dosage may be delivered in response to the determination of a difference between a currently measured heart failure state in a subject and a baseline heart failure state.

A measurement of the heart failure state of the heart includes, for example, a determination of ejection volume, by, for example, echocardiogram, a determination of other echocardiogram based measurements using relaxation parameters, such as, for example, E/E′, Edeceleration time, E/A ratio, and tau, and determinations of relaxation rates measured more invasively, such as, for example, tau, and −dP/dt.

In accordance with the present invention, an anti-fibrotic agent may be administered to the pericardial space in combination with the administration of one or more previously known treatment modalities, including, but not limited to, the pericardial administration of an additional therapeutic agent. As used herein, the term “additional therapeutic agent” represents one or more agents previously known to be effective for the treatment of a heart condition, including, but not limited to the treatment of atrial fibrillation or heart failure. In some embodiments of the present invention, the administration of an anti-fibrotic agent in combination with additional therapeutic agents may demonstrate therapeutic synergy. Likewise, the administration of two or more anti-fibrotic agents may demonstrate therapeutic synergy. As used herein, a combination may demonstrate therapeutic synergy if it is therapeutically superior to one or other of the constituents used individually. In some embodiments, a combination demonstrates therapeutic synergy if the efficacy of a combination is characterized as more than additive actions of each constituent.

Such an additional therapeutic agent is not an anti-fibrotic agent. The administration of the anti-fibrotic agent may take place before, during, and/or after the administration of the other mode of therapy. Such additional therapeutic agents include, but are not limited to, genetic agents, biologic agents, and pharmacologic agents. Suitable pharmacologic agents include, for example, agents that are angiotensin receptor blockers (ARB), including, but not limited to candesartan (Atacand), eprosartan (Teveten), irbesartan (Avapro), telmisartan (Micardis), valsartan (Diovan), losartan (Cozaar), and olmesartan (Benicar), agents that angiotensin converting enzyme (ACE) inhibitors, including, but not limited to, benazepril (Lotensin), captopril (Capoten), enalapril (Vasotec), fosinopril (Monopril), lisinopril (Prinivil, Zestril), moexipril (Univasc), perindopril (Aceon), quinapril (Accupril), ramipril (Altace) and trandolapril (Mavik), agents that improve cardiac contractility (including, but not limited to, digitalis drugs and adrenergic agonists), agents that suppress arrhythmias (including, but not limited to, class I, II, III, and IV agents and specialized drugs such as amiodarone), agents that dilate coronary arteries (including, but not limited to, nitroglycerin and calcium channel blockers), agents that lyse clots in the coronary circulation (including, but not limited to, thrombolytic agents such as streptokinase or tissue-type plasminogen activator (TPA)), agents that reverse symptoms of heart failure (including, but not limited to, beta-adrenergic blockers) and vasodilator, antiplatelet, anticoagulant, thrombolytic, anti-inflammatory, antiarrhythmic, initropic, antimitotic, angiogenic, and antiatherogenic agents.

In some embodiments of the present invention, additional technologies, such as, for example, implantable device technologies that measure, for example, pressure, activity, impedance, electrocardiography, temperature, and the like may be used. These technologies may communicate wirelessly to the delivery means to guide drug dosing and delivery in a closed-loop fashion.

With the present invention, one or more anti-fibrotic agents are delivered to the pericardial space of the heart. This targeted pericardial delivery of an anti-fibrotic agent of the present invention represents a major advance in the treatment of heart disease, including but not limited to heart failure and atrial fibrillation, ventricular fibrillation, and other arrhythmias, allowing for the delivery of therapeutic concentrations of anti-fibrotic agents directly to the heart and avoiding the sometimes serious side effects observed with the systemic delivery of such anti-fibrotic agents. As used herein “treating” or “treatment” can include therapeutic and/or prophylactic treatments. Desirable effects of treatment can include preventing occurrence or recurrence of disease, alleviation of symptoms, diminishment of any direct or indirect pathological consequences of the disease, decreasing the rate of disease progression, amelioration or palliation of the disease state, remission and/or improved prognosis.

The human heart wall consists of an inner layer of simple squamous epithelium, referred to as the endocardium, overlying a variably thick heart muscle or myocardium and is enveloped within a multi-layer tissue structure referred to as the pericardium. The innermost layer of the pericardium, referred to as the visceral pericardium or epicardium, clothes the myocardium. The epicardium reflects outward at the origin of the aortic arch to form an outer tissue layer, referred to as the parietal pericardium, which is spaced from and forms an enclosed sac extending around the visceral pericardium of the ventricles and atria. An outermost layer of the pericardium, referred to as the fibrous pericardium, attaches the parietal pericardium to the sternum, the great vessels and the diaphragm so that the heart is confined within the middle mediastinum. Normally, the visceral pericardium and parietal pericardium lie in close contact with each other and are separated only by a thin layer of a serous pericardial fluid that enables friction free movement of the heart within the sac. The space between the visceral and parietal pericardia is referred to as the pericardial space. In common parlance, the visceral pericardium is usually referred to as the epicardium, and epicardium will be used hereafter. Similarly, the parietal pericardium is usually referred to as the pericardium, and pericardium will be used hereafter in reference to parietal pericardium.

With the present invention, any of the wide variety of mechanisms for delivering an anti-fibrotic agent to the pericardial space available to one skilled in the art may be used, ranging, for example, from a simple puncture by means of a large bore needle to intricate catheter or cannula based systems.

For example, access to the pericardial space may be accomplished from outside the body by making a thoracic or subxiphoid incision to access and cut or pierce the pericardialsac. Access to the pericardial space from the exterior of the body, accomplished by passing a cannula or catheter type device through the chest wall and thereafter passing the cannula or catheter or a further instrument through the pericardium into the pericardial space, is disclosed, for example, in U.S. Pat. Nos. 5,336,252, 5,827,216, 5,900,433, 5,972,013, 6,162,195, 6,206,004, and 6,592,552. In certain cases the pericardial sac may be cut by a cutting instrument as disclosed, for example, in U.S. Pat. Nos. 5,931,810, 6,156,009, and 6,231,518.

Alternatively, an elongated perforating instrument device may be introduced from a skin incision or puncture by a transvenous or transarterial approach into the right or left heart chambers, respectively, and a cutting or piercing or penetrating mechanism at the distal end of the elongated perforating instrument may be operated to penetrate through the atrial or ventricular wall of the right heart chamber into the surrounding pericardial space without perforating the pericardial sac. For example, a transvenous catheter provided with a hollow helical needle adapted to rotate and pierce through the wall of a right or left heart chamber to access the pericardial space to deliver pharmacologic agents, as disclosed, for example, in U.S. Pat. No. 5,797,870, may be used. A transvenous catheter introduced into the right ventricular chamber to provide access through the right ventricular wall to enable passage of an electrical medical lead into the pericardial space, as disclosed, for example, in U.S. Pat. Nos. 4,991,578 and 5,330,496, may be used. It has also been proposed that a preferred site of penetration of catheters or electrical medical leads through the atrial wall into the pericardial space is within the right atrial appendage as disclosed, for example, in U.S. Pat. Nos. 5,269,326, 6,200,303, and 5,968,010. Transvenous approaches through either of the inferior vena cava or the superior vena cava are disclosed in these patents.

Further, any of the various methods and systems for accessing the pericardial space described in U.S. patent application Ser. No. 11/000,538, filed Dec. 1, 2004 (“Methods and Systems for Providing Therapies into the Pericardial Space”) may be used with the methods and systems of the present invention, for the intrapericardial delivery of an anti-fibrotic agent for the treatment of heart failure.

A single, acute dosage of an anti-fibrotic agent may be delivered, for example, by a subxyphoid approach. For such delivery, an anti-fibrotic agent may be in a formulation for controlled, sustained, or extended release. Many such suitable formulations are known, including, for example, polymeric or protein microparticle formulations.

Pericardial delivery of an anti-fibrotic agent may be, for example, by a catheter placed in the pericardial space. Such a catheter may be placed in the pericardium, for example, by an epicardial or a transarterial approach. For example, the pericardial space between a heart and its pericardium may be transvenously accessed to deliver an anti-fibrotic agent to the heart by passing a fixation catheter having a fixation catheter lumen extending between proximal and distal fixation catheter lumen openings and a distal tissue fixation mechanism through a selected peripheral vein and one of the inferior vena cava and the superior vena cava to establish a transvenous route into the right atrium of the heart; disposing the distal fixation mechanism and distal fixation catheter lumen opening proximate the right atrial wall; affixing the distal fixation mechanism to the right atrial wall; passing an infusion catheter through the fixation catheter lumen out of the distal fixation catheter lumen opening and through the stabilized atrial wall to dispose a distal catheter segment having a distal infusion catheter lumen end opening in the pericardial space; and delivering an anti-fibrotic agent through the infusion catheter to treat heart failure. In some aspects, the delivering step may include attaching a proximal connector of the infusion catheter to an infusion pump; subcutaneously implanting the infusion pump in the thoracic region; and operating the infusion pump to deliver the anti-fibrotic agent into the pericardial space. In some aspects, the operating step may include detecting a remotely transmitted therapy delivery command; and delivering a bolus of an anti-fibrotic agent.

A drug pump may be used for the delivery of an anti-fibrotic agent by a catheter to the pericardial space. Such a pump may be, for example, an external drug pump, such as, for example, the Medtronic® MiniMed® pump or an implantable drug pump, such as, for example, the Medtronic® SynchroMed Infusion System®. Systems for monitoring heart failure may be directly or indirectly (via wireless technology) coupled to the drug pump.

The present invention and/or one or more portions thereof may be implemented in hardware or software, or a combination of both. For example, the functions described herein may be designed in conformance with the principles set forth herein and implemented as one or more integrated circuits using a suitable processing technology, e.g., CMOS. As another example, the present invention may be implemented using one or more computer programs executing on programmable computers, such as computers that include, for example, processing capabilities, data storage (e.g., volatile and nonvolatile memory and/or storage elements), input devices, and output devices. Program code and/or logic described herein is applied to input data to perform functionality described herein and generate desired output information. The output information may be applied as an input to one or more other devices and/or processes, in a known fashion. Any program used to implement the present invention may be provided in a high level procedural and/or object orientated programming language to communicate with a computer system. Further, programs may be implemented in assembly or machine language. In any case, the language may be a compiled or interpreted language. Any such computer programs may preferably be stored on a storage media or device (e.g., ROM or magnetic disk) readable by a general or special purpose program, computer, or a processor apparatus for configuring and operating the computer when the storage media or device is read by the computer to perform the procedures described herein. The system may also be considered to be implemented as a computer readable storage medium, configured with a computer program, where the storage medium so configured causes the computer to operate in a specific and predefined manner to perform functions described herein. The present invention and/or one or more portions thereof include circuitry that may include a computer system operable to execute software to provide for the determination of a physiological state, e.g., heart failure. Software, one or more computer programs, data, input information, and/or output information, and/or portions thereof, may be saved in memory.

Although the circuitry may be implemented using software executable using a computer apparatus, other specialized hardware may also provide the functionality required to provide a user with information as to the physiological state of the individual. As such, the term circuitry as used herein includes specialized hardware in addition to or as an alternative to circuitry such as processors capable of executing various software processes. The computer system may be, for example, any fixed or mobile computer system, e.g., a personal computer or a minicomputer. The exact configuration of the computer system is not limiting and most any device capable of providing suitable computing capabilities may be used according to the present invention. Further, various peripheral devices, such as a computer display, a mouse, a keyboard, memory, a printer, etc., are contemplated to be used in combination with a processing apparatus in the computer system. In view of the above, it will be readily apparent that the functionality as described herein may be implemented in any manner as would be known to one skilled in the art.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Relative Efficacy of Relaxin and Valsartan for Reversing Left Atrial and Ventricular Fibrosis in Spontaneously Hypertensive Rats

Fibrosis is a key pathologic associate of atrial fibrillation (left atrial fibrosis) and diastolic heart failure (left ventricular fibrosis). Reversing pathological fibrosis in these clinical entities represents a large unmet treatment need. With the present example, the pericardial space us utilized as a drug delivery device, allowing high dose therapy targeted at the heart with little systemic exposure. This example will examine and compare the efficacy of human recombinant relaxin (RLX) and the angiotensin receptor blocker (ARB) valsartan for reversing established cardiac (left atrial, ventricular) fibrosis and left ventricular diastolic dysfunction. This example will also compare the efficacies of systemic and intrapericardial routes of drug delivery.

The spontaneously hypertensive (SHR) rats are generally used for studies in hypertension and cardiovascular research (see, for example, the worldwide web at informatics.jax.org/external/festing/rat/docs/SHR.shtml) and the SHR rat is well characterized as an outbred model of established left atrial and ventricular cardiac fibrosis and left ventricular diastolic dysfunction. The Wistar Kyoto Outbred rat (WKY) that will be used as a non-hypertensive control is the litter mate of the SHR strain. SHR and WKY rats will be obtained from Taconic (Hudson, N.Y.).

With this example, twelve groups, each with 10 animals (in anticipation of inter-individual variation), will be tested. There will be six groups of SHR animals and a mirror cohort of WKY animals. The groups will be as follows:
Group 1: systemic (subcutaneous) placebo therapy
Group 2: systemic RLX therapy
Group 3: systemic ARB therapy
Group 4: intrapericardial placebo therapy
Group 5: intrapericardial RLX therapy
Group 6: intrapericardial ARB therapy

It is expected that two weeks of RLX and ARB therapy will decrease atrial and ventricular fibrosis (as measured by collagen content determined by histological and biochemical methods) in myogenic activity in rats. In previous studies of cardiac fibrosis prevention with the administration of other agents in the SHR model, the standard deviation was large. Based on this and in anticipation of a pre-completion mortality rate within groups of 20%, ten rats per group is reasonable to support detection of significant differences at 80% at a p value of 0.05%.

At baseline, each animal will undergo implantation of a subcutaneous minipump. Animals will be administered an injection of Pentobarbital to produce an adequate plane of anesthesia. The back of the animal is shaved and cleaned with betadine prep solution. A small incision will be made along the back of the animal using sterile instruments. Using small blunt scissors, the soft tissue is separated from the skin making a pocket for the minipump. The pump will be connected to either a subcutaneous catheter or an intrapericardial catheter. The subcutaneous catheter will be placed local to the pump. The intrapericardial catheter will be tunnelled to the pump pocket from the thorax, where it will be inserted into the pericardial space via suprastemal thoracotomy and held in place using prolene suture. The thorax will be closed using absorbable suture. Once the minipump is inserted, the skin will be pulled over the pump and the pump pocket closed using absorbable suture. The animal will be observed to make sure it is ambulatory and alert before being returned to regular housing. Six groups of SHR and six mirror groups of WKY rats will be studied (10 animals/group). Treatment duration will be two weeks for all animals. The vehicle for administration of RLX will be 20 mmol sodium acetate, ph 5.0.

Group 1 will receive systemic (subcutaneous) placebo therapy.

Group 2 will receive systemic RLX therapy at 0.5 mg/kg/day.

Group 3 will receive systemic ARB (valsartan) therapy at 1 mg/kg/day.

Group 4 will receive intrapericardial placebo therapy.

Group 5 will receive intrapericardial RLX therapy at 0.5 mg/kg/day.

Group 6 will receive intrapericardial ARB (valsartan) therapy at 1 mg/kg/day.

After two weeks, animals will undergo transthoracic echocardiography and tail blood pressure cuff measurements. After these are completed, and under the same anesthetic plane, the right neck of the animals will then be shaved and surgical cutdown for the carotid artery will be performed. Specifically, the mouse will be placed in a supine position on a 37° C. pad under the surgical microscope and its limbs will be restrained with tape. A 0.5 cm skin incision will be performed in the right neck area and the carotid artery will be isolated using 0-silk sutures. The cranial aspect of the carotid artery will be ligated and a microsurgical clip will be placed on the proximal carotid artery for hemostasis. An arteriotomy will be performed with microsurgical scissors, and a 1.4 French conductance catheter (Millar, Inc, Houston, Tex.) will be introduced into the carotid artery and advanced retrograde across the aortic valve into the left ventricle. The catheter will be advanced under continuous hemodynamic monitoring to insure proper placement in the LV. The catheter will be secured within the carotid artery with the proximal suture. A 0.5 cm longitudinal abdominal incision will be made and blunt dissection used to expose the inferior vena cava. Total estimated time to completion of placement of conductance catheter in LV and completion of abdominal incision is 10-15 minutes. LV pressure-volume loops will be recorded in the steady state and during IVC compression, performed with a 6-0 silk snare suture with a total estimated time to completion of 20 minutes, after which they will be killed and the heart removed for histological and biochemical analyses. Experimental endpoint will be 14 days. Blood will be taken at sacrifice for measurement of serum rhRLX by ELISA using an abdominal aortic puncture following anesthesia.

The histological and biochemical analyses of heart tissue may include, but is not limited to, an analysis of collagen content by hydroxyproline assay, gel electrophoresis, and quantitative histology; a determination of matrix metalloproteinase (MMP) expression by zymography; a determination of proliferating cell nuclear antigen (PCNA) and a-smooth muscle actin (a-SMA) myofibroblast expression by western blotting; a determination of cardiac hypertrophy by measuring myocyte cell size and real-time polymerase chain reaction (PCR) of associated genes. These determination will be as described herein and by methods known to one of ordinary skill in the art, for example, as described in more detail in Lekgabe et al., 2005, Hypertension; 46:412-418.

The complete disclosure of all patents, patent applications, and publications, and electronically available material (including, for instance, nucleotide sequence submissions in, e.g., GenBank and RefSeq, and amino acid sequence submissions in, e.g., SwissProt, PR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

It is claimed:

1. A method of treating atrial fibrillation in a subject, comprising administering systemically a therapeutically effective amount of relaxin to a subject with atrial fibrillation.

2. The method of claim 1, wherein administering a therapeutically effective amount of relaxin to the subject comprises administering 0.5 mg/kg/day relaxin to the subject.

3. The method of claim 1, wherein administration of the relaxin reduces atrial fibrosis in the subject.

4. The method of claim 1, further comprising selecting the subject with atrial fibrillation.

5. The method of claim 1, wherein administering systemically the therapeutically effective amount of relaxin comprises intravenous administration of the relaxin.

6. The method of claim 1, wherein the subject is a human subject.

7. The method of claim 1, further comprising detecting atrial fibrillation of the heart in the subject.
